(19) United States
(12) Patent Application Publication
(10) Pub. No.: US 2021/0123038 A1
(43) Pub. Date: Apr. 29, 2021

(54) Acoustic Transducer Automated Start and Run

(71) Applicant: FloDesign Sonics, Inc., Wilbraham, MA (US)

(72) Inventors: Ronald E. Musiak, Westfield, MA (US); John Artis, Santa Rosa, CA (US); Bart Lipkens, Bloomfield, CT (US)

(21) Appl. No.: 16/633,504

(22) PCT Filed: Jul. 26, 2018
(86) PCT No.: PCT/US2018/043991

§ 371 Date: Jan. 23, 2020

Related U.S. Application Data
(60) Provisional application No. 62/537,438, filed on Jul. 26, 2017.

(51) Int. Cl. C12N 13/00; C02F 1/36; C02F 1/00; C02F 1/40; B01D 17/02; B01D 17/12; C12M 1/00; B06B 1/02

(52) U.S. Cl. CPC C12N 13/00; C02F 1/36; C02F 1/008; C02F 1/40; B06B 2201/77; B01D 17/12; C12M 47/02; B06B 1/0261; B06B 2201/55; B01D 17/02

(57) ABSTRACT

An operating point for control of an acoustic transducer can drift during operation and be compensated. A model for the transducer and/or environment frequency response is provided and used to compensate feedback from the transducer to determine an adjustment for the operating point. The model can be recalibrated during operation.

### STATEMENT REGARDING FEDERALLY SPONSORED RESEARCH OR DEVELOPMENT

(Not Applicable)

### BACKGROUND

An acoustic transducer with a piezoelectric element can be used to generate an acoustic wave. When propagating in a host fluid, the acoustic wave can exert forces on particles or secondary fluid droplets (referred to herein collectively as particles) contained in the host fluid when there is a differential in density and/or compressibility, otherwise known as the acoustic contrast factor. The pressure profile in an acoustic wave contains areas of local minimum pressure amplitudes at nodes and local maxima at anti-nodes of a sinusoidal acoustic wave. Depending on their density and compressibility, the particles can be trapped at the nodes or anti-nodes of the acoustic wave.

The piezoelectric element that can generate the acoustic wave can be electrically excited. Such an electrical excitation represents a complex loading on an electrical driver that drives the piezoelectric element.

### SUMMARY

An acoustic transducer can be driven to create an acoustic wave, which can be a traveling wave or a standing wave, in an adjacent fluid that has at least non-zero acoustic forces in a direction transverse to the propagation direction of the wave. The fluid can be contained in an acoustic resonance chamber, the combination of the acoustic transducer and acoustic resonance chamber being referred to herein as an acoustic system. The acoustic wave that generates the acoustic forces in the fluid in more than one dimension is referred to herein as a multi-dimensional acoustic wave. The multi-dimensional acoustic wave generation process takes advantage of the higher-order vibratory modes of a loosely suspended piezoelectric material, which may be implemented, for example, in the form of a plate.

Control of the acoustic transducer can be implemented on the basis of setpoints. For example, a user can set a desired power level for power delivered to the transducer. Performance of acoustophoresis in an acoustic chamber using the acoustic transducer can be modulated on the basis of modulated input power to the acoustic transducer. In some instances, a power setpoint is desired for operation, while other parameters, such as frequency, phase, voltage or current, for example, are modified. The power setpoint determines the power output of an RF power supply or power amplifier. A power control is provided to maintain the power setpoint, while other parameters associated with operation of the acoustophoresis device are varied. The power control senses signals provided to the acoustic transducer, such as, for example, voltage and current. These feedback signals are used to determine frequency and phase angle for the power delivered to the transducer. In some examples, a buck converter is used as the power supply. The buck converter has a response bandwidth, which may influence the responsiveness of the power control. For example, if the buck converter bandwidth is relatively narrow, the system response for the power control may be relatively slow for the desired operational performance environment for the acoustophoresis device.

The acoustic system can be initialized to prepare for continuous operation. The initialization process can include: identifying the material that is to be input to and/or processed by the acoustic system, choosing a configuration for operation of the acoustic system, inputting characteristics of the acoustic system, calibrating the acoustic system, initializing the acoustic system and/or operating the acoustic system in a continuous mode.

A number of different materials may be processed through the acoustophoresis device, each of which may provide different load characteristics on the acoustic transducer and acoustic chamber. The power supply thus may be subjected to a wide range of loads, which may place demands on the power supply that are challenging to meet. For example, heavy loading of the acoustic transducer and/or acoustic chamber experienced with certain types of materials being processed may cause power supply components to be overloaded, and/or overheated, or may cause trip point thresholds to be met or exceeded. The heavy loading or trip point thresholds crossings may cause faults to be identified in the power control, causing the power supply to be shut down. In addition, the power demands on the power supply may change significantly with changes in other operational parameters, such as temperature, frequency or loading characteristics, including reactance. Power control based on a desired power levels the point may thus imply other operational setpoints, such as frequency, to manage operation of the power supply and acoustophoresis device to handle a range of loads.

The characteristics of the input to the piezoelectric material of the acoustic transducer can be modified to permit various vibration modes of the piezoelectric material. For example, a pure sine wave can induce a very succinct vibration of the piezoelectric material, while a signal with harmonic content can cause parasitic vibrations of the piezoelectric material. The input to the piezoelectric material may influence the heat generated or input into the fluid in which the acoustic wave is formed. The input may generate more complicated motion in the fluid coupled with the piezoelectric material.

Piezoelectric material changes shape based on an electrical signal applied to it, such as a voltage or current signal, or based on a corresponding electric field permeating the material. The electric field from external charges affects the fields of the bound charges in the material and thereby affects the shape of the material. The electrical signal can be from a voltage source. In that case the amount of material deformation is related to the voltage applied. For example, the deformation may be voltage clamped or voltage damped. The amount of charge induced is related to the applied voltage and the properties of the material. This relationship can be expressed mathematically as Q=C*V, where Q is charge, C is material capacitance, and V is the voltage of the applied signal. Electrodes may be attached to the piezoelectric material to provide a conduit for the applied signal. In that case the voltage, and the corresponding electric field, is a function of the externally applied charges. Using the above equation, the voltage can be express as V=Q/C. The resultant voltage may be unconstrained in relation to operation of the piezoelectric device. The C of the piezoelectric device is due to its physical geometry and material properties. Since the material changes shape as a function of the electric field permeating it, the C of the device is a function of the electric field permeating it. For a given Q, and driving the material with a current source that is a time varying source of charge, C changes as a function of electric field, which changes the voltage across the device to accommodate the changed C. In a voltage driven system, the electric field can determine the amount of charge, which can determine the degree of deformation and correspondingly the amount of change in C. To encourage multimode behavior in piezoelectric material, the piezoelectric material can be configured to be free floating, and in some examples, is made to be as free floating as possible in both a mechanical and electrical sense.

The control of the multi-dimensional acoustic wave and the acoustic resonator or transducer is an important part of an acoustophoresis process. For example, as a multi-dimensional acoustic wave is utilized to trap biologic cells and cell debris from a bioreactor process, the reactance of the resonator changes. By sensing the voltage and current of the RF transmission line to the piezoelectric element generating the multi-dimensional acoustic wave, the resonator can be properly tuned to optimize the acoustophoresis process. The reactance and power can be extracted from the voltage and current signals on the piezoelectric element. For example, voltage and current signals can be provided to a digital signal processor (DSP), which can be used to calculate RF reactance and power. The measured and calculated parameters of operation for the piezoelectric element can be used to provide feedback for the tuning process. This tuning process may consist of adjusting the gain of the amplifier to achieve a desired power that is provided to the piezoelectric element and/or adjusting the frequency of the drive signal to achieve a desired reactance of the resonator, as examples.

The multi-dimensional acoustic wave is generated through a multimode perturbation of the piezoelectric material by electronic signal generated by a function generator or oscillator and modified by an amplifier. The generation of the multi-dimensional acoustic wave and the multimode perturbation of the piezoelectric material is described in U.S. Pat. No. 9,228,183, which is incorporated herein by reference.

An RF power driver is provided to drive the acoustic transducer. In some implementations, the power driver is composed of a DC-DC converter, which may be a buck converter, boost converter or buck-boost converter, coupled to a DC-AC inverter. A filter is provided between the converter and inverter. The output of the inverter may be supplied to an LCL matching filter to produce a DC signal that the inverter can use. In such an example implementation, the filter may impose constraints on or otherwise control the system response time.

A control, which may be a digital or analog control, is provided that can receive inputs fed back from the acoustic transducer or other system components and provide control signals to various components of the RF power driver. The control can provide control signals to vary the DC output of the converter, and/or modify and control the frequency, phase, amplitude of the power, voltage and/or current of the drive signal for the acoustic transducer. Control signals provided by the control can vary the operation of the inverter to modify and control the frequency of the drive signal. The RF power driver with the control permits control and modulation of the acoustic transducer as a highly reactive load, while maintaining desired transducer and acoustic chamber performance.

A control technique provides a system and method for locating desired operating points for an acoustic transducer-cavity combination, with or without loading, which loading may be highly reactive. Feedback from the acoustic transducer can be used to locate the resonance and anti-resonance frequencies of transducer operation.

According to some implementations, an operating frequency less than the transducer anti-resonance is inspected for minimum reactance as a point of operation. Some implementations locate a frequency above the anti-resonance frequency, which frequency is inspected for maximum reactance as a point of operation. The frequency of the drive signal can be controlled and/or modified to be set to a point of minimum reactance that is below the anti-resonance frequency. A number of minima exist in the frequency range below anti-resonance, any of which can be used for a frequency operating setpoint. According to these implementations, a desired level of efficiency can be obtained for acoustophoresis using the acoustic transducer to generate an acoustic wave through fluid in the acoustic chamber or cavity to which the transducer is coupled. The points of operation that are determined according to a control technique discussed herein can be frequency setpoints, which can be dynamically maintained. For example, a desired point of operation may change with characteristics of operation of the acoustic chamber, such as a volume of material entrained in the fluid, a degree of material separation, temperature, power delivered to the transducer, and other phenomena that may influence or modify a desired operating point.

### BRIEF DESCRIPTION OF THE SEVERAL VIEWS OF THE DRAWINGS

The disclosure is described in greater detail below, with reference to the accompanying drawings, in which:

FIG. 1 is a diagram showing an acoustic chamber and connections thereto;

FIG. 2 is a diagram illustrating acoustophoresis with an acoustic transducer and reflector;

FIG. 3 is a cross-sectional side view of an acoustic transducer with a free piezoelectric element;

FIG. 4 is a cross-sectional view of an acoustic transducer with a damped piezoelectric element;

FIG. 5 is a graph illustrating force applied to a particle in a fluid;

FIG. 6 is a graph illustrating impedance of a piezoelectric element;

FIG. 7A is a diagram illustrating different vibrational modes for an acoustic transducer;

FIG. 7B is an isometric view of an acoustic chamber;

FIG. 7C is a left side elevation view of the acoustic chamber in FIG. 7B;

FIG. 7D is a front elevation view of the acoustic chamber in FIG. 7B;

FIG. 8 is a graph illustrating transducer frequency responses and frequencies with dominant modes;

FIG. 9 is a graph illustrating a frequency response for an acoustic transducer;

FIG. 10 is a graph illustrating a frequency response for an acoustic transducer;

FIG. 11 is a block diagram illustrating a control technique for an acoustic transducer;

FIG. 12 is a graph illustrating power, reactance, resistance and peak performance for an acoustic transducer;

FIG. 13 is a flowchart illustrating a process for determining compensation model parameters;

FIG. 14 is a flowchart illustrating a process for locating minimum reactances;

FIG. 15 is a flowchart illustrating a process for tracking changes in operational parameter combinations;

FIG. 16 is a flowchart illustrating an automated startup and run control process for an acoustic separation system; and

FIG. 17 is a flowchart illustrating an automated startup and run control process for an acoustic separation system.

### DETAILED DESCRIPTION

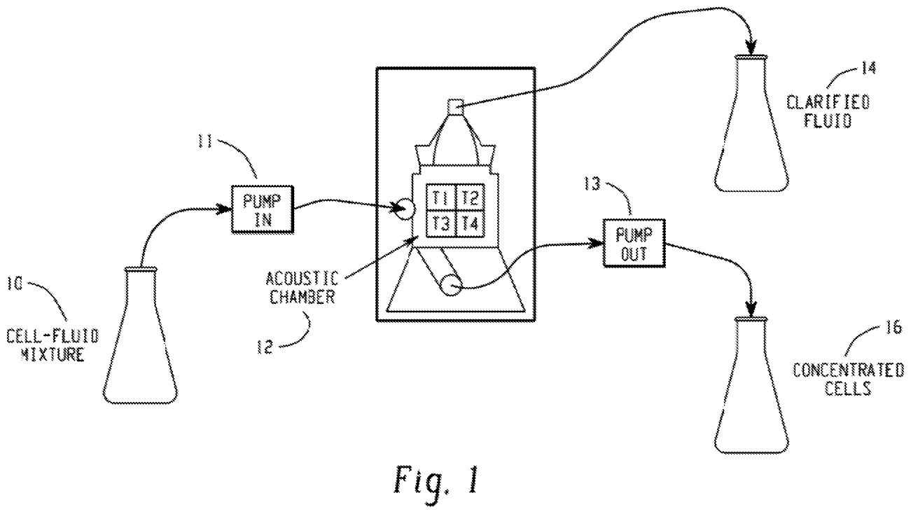

FIG. 1 is a broad overview of an acoustic wave separator system. A mixture 10 of a host fluid and a secondary phase (e.g. particles, cells, or a second different fluid) is sent via a pump 11 into an acoustic chamber 12. Here, the mixture is a cell-fluid mixture. In the acoustic chamber, the secondary phase is concentrated out of the host fluid. The concentrated cells 16 are sent by another pump 13 to be collected. The host fluid, which is more clarified due to the removal of the concentrated cells, is separately collected (indicated by reference numeral 14). Generally speaking, the acoustic chamber has at least one inlet and at least one outlet.

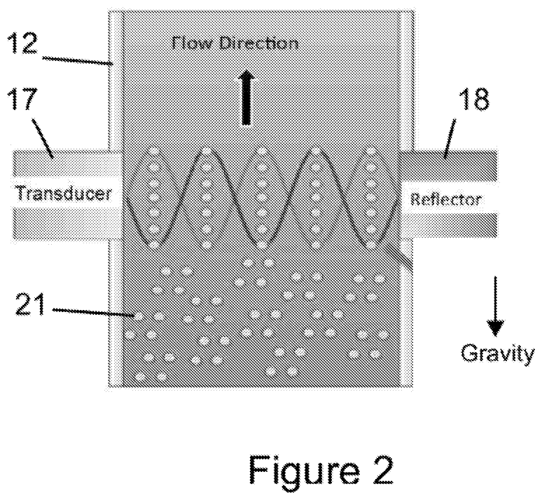

The acoustic chamber operates as shown in FIG. 2. One or more multi-dimensional acoustic waves are created between an ultrasonic transducer 17 and a reflector 18. The acoustic wave is illustrated as beginning and ending with local minima, however, other implementations are possible. For example, the acoustic wave can be offset at the transducer or the reflector so that local minima or maxima are spaced from the transducer or from the reflector. The reflected wave (or wave generated by an opposing transducer) can be in or out of phase with the transducer generated wave. The characteristics of the acoustic wave can be modified and/or controlled by the drive signal applied to the transducer, such as by modifying and/or controlling the power, voltage, current, phase, amplitude or frequency of the drive signal. Acoustically transparent or responsive materials may also be used with the transducer or reflector to modify and/or control the acoustic wave.

As the fluid mixture flows through acoustic chamber 12 with ultrasonic transducer 17 active, particles 21 cluster, collect, agglomerate, aggregate, clump, or coalesce at the nodes or anti-nodes of the multi-dimensional acoustic wave, depending on the particles' or secondary fluid's acoustic contrast factor relative to the host fluid. The particles form clusters that eventually exit the multi-dimensional acoustic wave nodes or anti-nodes when the clusters have grown to a size large enough to overcome the holding force of the multi-dimensional acoustic wave (e.g. coalescence or agglomeration increases the gravity or buoyancy forces on the clusters to the point of overcoming drag and/or acoustic forces). For fluids/particles that are more dense than the host fluid (such as the cells of FIG. 1), the clusters sink to the bottom and can be collected separately from the clarified host fluid. For fluids/particles that are less dense than the host fluid, the buoyant clusters float upwards and can be collected.

The multi-dimensional acoustic wave generates acoustic radiation forces, which act as three-dimensional trapping fields. The acoustic radiation force is proportional to the particle volume (e.g. the cube of the radius) when the particle is small relative to the wavelength. The force is proportional to frequency and the acoustic contrast factor. The force scales with acoustic energy (e.g. the square of the acoustic pressure amplitude). When the acoustic radiation force exerted on the particles is stronger than the combined effect of fluid drag force and buoyancy and/or gravitational force, the particles are trapped within the acoustic wave. The particle trapping in a multi-dimensional acoustic wave results in clustering, concentration, agglomeration and/or coalescence of the trapped particles. Relatively large solids or fluids of one material can thus be separated from smaller particles of a different material, the same material, and/or the host fluid through enhanced gravitational/buoyancy separation.

The multi-dimensional acoustic wave generates acoustic radiation forces in both the axial direction (e.g., in the direction of propagation of the acoustic wave, between the transducer and the reflector, which may be at an angle across a direction of flow, and in some instances may be perpendicular to the flow direction) and the lateral direction (e.g., in the flow direction or transverse to the direction between the transducer and the reflector). As a mixture of host fluid and particles flows through the acoustic chamber, particles in suspension experience a strong acoustic force driving the particles to locales of lower acoustic pressure, resulting in clustering, agglomeration or clumping. The axial and lateral acoustic radiation forces can combine or individually contribute to overcoming fluid drag for such clumps of particles, to continually grow the clusters, which can exit the mixture due to gravity or buoyancy. As the particle cluster increases in size, the drag forces per particle decreases. In addition, as the particle cluster grows in size, the acoustic radiation force per particle decreases, which may lead to more rapid dropout of the clusters from the acoustic wave. The lateral force component and the axial force component of the multi-dimensional acoustic wave can be controlled by the drive signal provided to the acoustic transducer to be within a same or different order of magnitude. The lateral force of a multi-dimensional acoustic wave that is generated by an acoustic transducer as discussed herein is much higher than the lateral force of a planar wave, usually by two orders of magnitude or more.

Particle drag and acoustic radiation force effects may influence optimal operation of the systems and methods of the present disclosure. At low Reynolds numbers of less than 10, laminar flow dominates, and viscous forces are much stronger than inertial forces.

As the particles are trapped by the multi-dimensional ultrasonic acoustic wave, they begin to aggregate and form a clump of particles. The drag on this clump of particles is a function of the geometry of the clump and is not merely the sum of the drag of the individual particles that make up the clump.

For laminar flow, the Navier Stokes equation is expressed as:

$$ρ ( \frac{\partial V}{\partial t} + ( V \cdot ∇ ) V ) ) = - ∇ P + μ {∇}^{2} V$$

where

$$\frac{\partial V}{\partial t}$$

represents unsteady motion, (V·∇)V) represents inertial motion, −∇P represents pressure motion, and μ∇²V represents viscous motion.

For low Reynolds numbers, the unsteady motion and inertial motion terms can be ignored (i.e. set equal to zero), and the equation can be simplified to:

∇P=μ∇²V

For a particle of diameter a, the following equations hold:

$$∇ P ∝ μ \frac{V}{a} F = 6 πμ aV$$

where P is pressure, μ is the dynamic viscosity, a is the particle diameter, V is the flow velocity, and F is the Stoke's drag.

The multi-dimensional acoustic wave used for particle collection is obtained by driving an ultrasonic transducer composed of a piezoelectric material at a frequency that generates the acoustic wave and excites a fundamental 3D vibration mode of the transducer. These vibration modes can be described as Bessel functions. The transducer may be composed of various materials that may be perturbed to generate an ultrasonic wave. For example, the transducer may be composed of a piezoelectric material, including a piezoelectric crystal or poly-crystal or ceramic crystal. The piezoelectric material in the transducer may sometimes be referred to herein as PZT, which is derived from the industry term of PZT-8, which is a piezoelectric material made of lead zirconate titanate. The piece of piezoelectric material in the ultrasonic transducer is sometimes referred to herein as a crystal. The piezoelectric material in the ultrasonic transducer can be electrically excited or perturbated to achieve a multimode response, which can generate a multi-dimensional acoustic wave. A piezoelectric material can be specifically designed to deform in a multimode response at designed frequencies, which can generate multi-dimensional acoustic waves with designed characteristics. The multi-dimensional acoustic wave may be generated with distinct modes of the piezoelectric material such as a 3×3 mode that generates multi-dimensional acoustic waves. A multitude of multi-dimensional acoustic waves may also be generated by allowing the piezoelectric material to vibrate through many different mode shapes. Thus, the material can be selectively excited to operate in multiple modes such as a 0×0 mode (i.e. a piston mode), 1×1, 2×2, 1×3, 3×1, 3×3, and other higher order modes. The material can be operated to cycle through various modes, in a sequence or skipping past one or more modes, and not necessarily in a same order with each cycle. This switching or dithering of the material between modes allows for various multi-dimensional wave shapes, along with a single piston mode shape to be generated over a designated time.

The crystal may have a major dimension on the order of 1 inch and larger. The resonance frequency of the crystal may nominally be about 2 MHz, and may be operated at one or more frequencies. Each ultrasonic transducer module can be implemented with one crystal or multiple crystals that each act as a separate ultrasonic transducer. Each crystal can be excited or driven (controlled) by one or multiple drivers or controllers, which drivers or controllers may include signal amplifiers. The crystal can be square, rectangular, irregular polygon, or generally of any arbitrary shape. The transducer(s) is/are used to create a pressure field that generates forces of the same order of magnitude in a direction of propagation of the acoustic wave (axial), and in a direction to the side or transverse to the axial direction (lateral).

Backing layers have been added to crystals to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

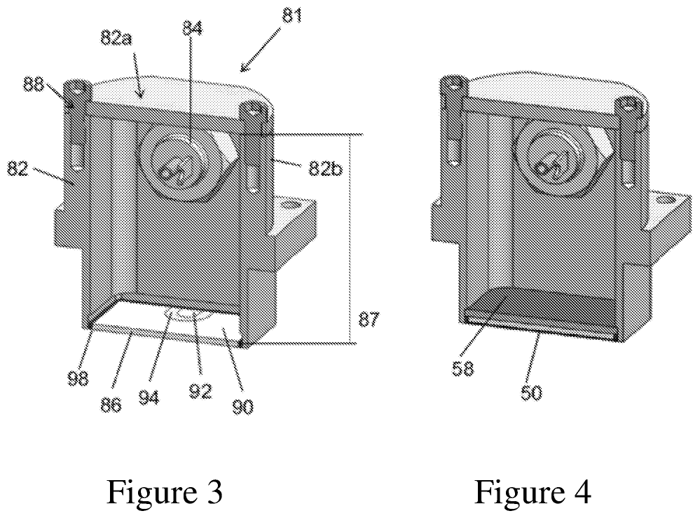

FIG. 3 is a cross-sectional view of an ultrasonic transducer 81 according to an example of the present disclosure. Transducer 81 is shaped as a disc or a plate, and has an aluminum housing 82. The piezoelectric crystal is a mass of perovskite ceramic crystals, each consisting of a small, tetravalent metal ion, usually titanium or zirconium, in a lattice of larger, divalent metal ions, usually lead or barium, and O2- ions. As an example, a PZT (lead zirconate titanate) crystal 86 defines the bottom end of the transducer, and is exposed from the exterior of the housing. The crystal has an interior surface and an exterior surface. The crystal is supported on its perimeter by a small elastic layer 98, e.g. silicone or similar material, located between the crystal and the housing. Put another way, no wear layer is present. In particular embodiments, the crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT crystal 86 is connected to a positive electrode 90 and a negative electrode 92, which are separated by an insulating material 94. The electrodes can be made from any conductive material, such as silver or nickel. Electrical power is provided to the PZT crystal 86 through the electrodes on the crystal. Note that the crystal 86 has no backing layer or epoxy layer. Put another way, there is an air gap 87 in the transducer between aluminum top plate 82a and the crystal 86 (i.e. the housing is empty). A minimal backing 58 (on the interior surface) and/or wear plate 50 (on the exterior surface) may be provided in some embodiments, as seen in FIG. 4.

The transducer design can affect performance of the system. A typical transducer is a layered structure with the ceramic crystal bonded to a backing layer and a wear plate. Because the transducer is loaded with the high mechanical impedance presented by the acoustic wave, the traditional design guidelines for wear plates, e.g., half wavelength thickness for acoustic wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes (i.e. near eigenfrequency) with a high Q-factor. The vibrating ceramic crystal/disk is directly exposed to the fluid flowing through the acoustic chamber.

Removing the backing (e.g. making the crystal air backed) also permits the ceramic crystal to vibrate at higher order modes of vibration with little damping (e.g. higher order modal displacement). In a transducer having a crystal with a backing, the crystal vibrates with a more uniform displacement, like a piston. Removing the backing allows the crystal to vibrate in a non-uniform displacement mode. The higher order the mode shape of the crystal, the more nodal lines the crystal has. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, from contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymers or polymer films. Organic and biocompatible coatings such as silicone or polyurethane are also usable as a wear surface.

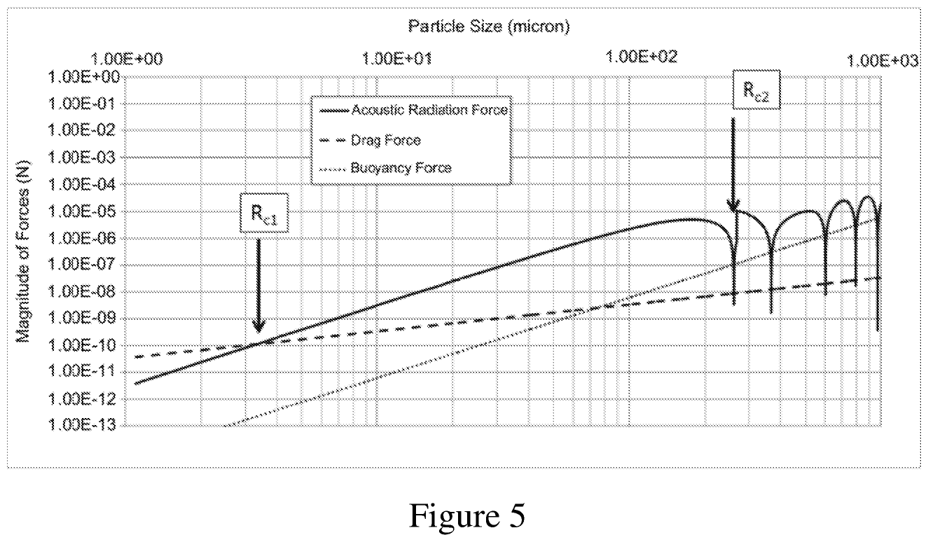

FIG. 5 is a log-log graph (logarithmic y-axis, logarithmic x-axis) that shows the scaling of the acoustic radiation force, fluid drag force, and buoyancy force with particle radius, and provides an explanation for the separation of particles using acoustic radiation forces. Although FIG. 5 illustrates relationships with buoyancy forces, the relationships shown are substantially similar for gravity forces on particles. Accordingly, buoyancy and gravity forces are discussed herein as applicable.

The buoyancy force is a particle volume dependent force, and is therefore negligible for particle sizes on the order of micron, but grows, and becomes significant for particle sizes on the order of hundreds of microns. The fluid drag force (Stokes drag force) scales linearly with fluid velocity, and therefore typically exceeds the buoyancy force for micron sized particles, but is negligible for larger sized particles on the order of hundreds of microns. The acoustic radiation force scaling and characteristics are different from the fluid drag force. When the particle size is small, Gor'kov's equation is accurate and the acoustic trapping force scales with the volume of the particle. Eventually, when the particle size grows, the acoustic radiation force no longer increases with the cube of the particle radius, and will rapidly vanish at a certain critical particle size. For further increases of particle size, the radiation force increases again in magnitude but with opposite phase (not shown in the graph). This pattern repeats for increasing particle sizes.

Initially, when a suspension is flowing through the system with primarily small micron sized particles, the acoustic radiation force balances the combined effect of fluid drag force and buoyancy force to permit a particle to be trapped in the acoustic wave. In FIG. 5 this trapping happens at a particle size labeled as Li. The graph indicates that as particle size increases, the larger particles experience greater acoustic force, and particles larger than R_{c1} are also trapped. As smaller particles are trapped in the acoustic wave, particle clustering/coalescence/clumping/aggregation/agglomeration takes place, resulting in continuous growth of effective particle size. As particles cluster, the total drag on the cluster is reduced compared to the sum of the drag forces on the individual particles. In essence, as the particles cluster, they shield each other from the fluid flow and reduce the overall drag of the cluster. As the particle cluster size grows, the acoustic radiation force reflects off the cluster, such that the net acoustic radiation force decreases per unit volume. The acoustic lateral forces on the particles may be larger than the drag forces for the clusters to remain stationary and grow in size.

Particle size growth continues until the buoyancy force becomes dominant, which is indicated by a second critical particle size, R_{c2}. The buoyancy force per unit volume of the cluster remains constant with cluster size, since it is a function of the particle density, cluster concentration and gravity constant. Therefore, as the cluster size increases, the buoyancy force on the cluster increases faster than the acoustic radiation force. At the size R_{c2}, the particles will rise or sink, depending on their relative density with respect to the host fluid. At this size, acoustic forces are secondary, gravity/buoyancy forces become dominant, and the particles naturally drop out or rise out of the acoustic wave. Some particles may remain in the acoustic wave as clusters of others drop out, and those remaining particles and new particles entering the acoustic chamber with the flow of a fluid mixture continue to move to the three-dimensional nodal locations, repeating the growth and drop-out process. As particle cluster size continues to increase in the graph of FIG. 5 beyond R_{c2}, a periodic rapid decrease in acoustic radiation force is observed. This rapid decrease represents the cluster size reaching a size equivalent to a half-wavelength interval, where the cluster begins to overlap the node or anti-node of the acoustic wave. This phenomenon explains the quick drops and rises in the acoustic radiation force beyond size R_{c2}. Thus, FIG. 5 explains how small particles can be trapped continuously in an acoustic wave, grow into larger particles or clumps, and rise or settle out because of buoyancy/gravity forces overcoming drag/acoustic forces.

In some examples, the size, shape, and thickness of the transducer can determine the transducer displacement at different frequencies of excitation. Transducer displacement with different frequencies may affect particle separation efficiency. Higher order modal displacements can generate multi-dimensional acoustic waves with strong gradients in the acoustic field in all directions, thereby creating strong acoustic radiation forces in all directions, which forces may, for example, be equal in magnitude, leading to multiple trapping lines, where the number of trapping lines correlate with the particular mode shape of the transducer.

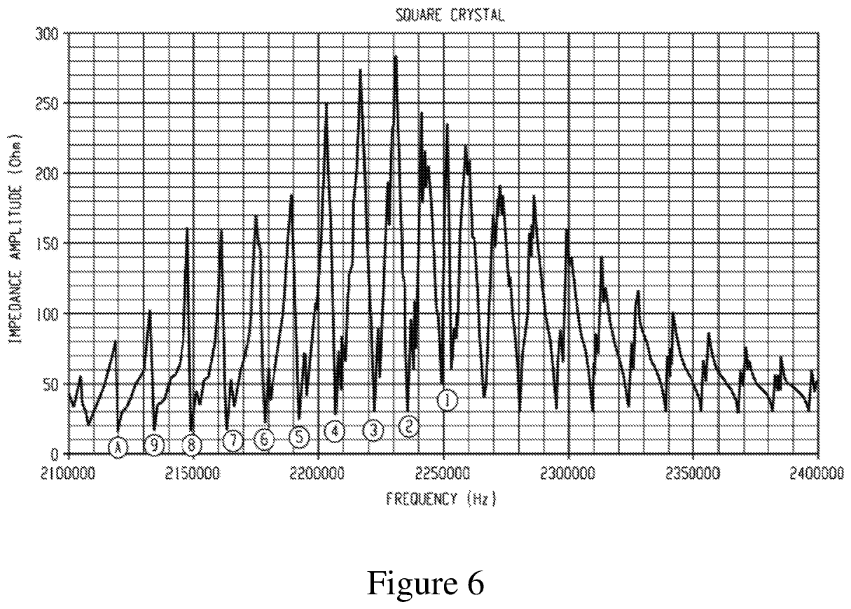

FIG. 6 shows the measured electrical impedance amplitude of the transducer as a function of frequency in the vicinity of the 2.2 MHz transducer resonance. The minima in the transducer electrical impedance correspond to acoustic resonances of a water column and represent potential frequencies for operation. Numerical modeling has indicated that the transducer displacement profile varies significantly at these acoustic resonance frequencies, and thereby directly affects the acoustic wave and resulting trapping force. Since the transducer operates near its thickness resonance, the displacements of the electrode surfaces are essentially out of phase. The typical displacement of the transducer electrodes may not be uniform and varies depending on frequency of excitation. Higher order transducer displacement patterns result in higher trapping forces and multiple stable trapping lines for the captured particles.

To investigate the effect of the transducer displacement profile on acoustic trapping force and particle separation efficiencies, an experiment was repeated ten times, with all conditions identical except for the excitation frequency. Ten consecutive acoustic resonance frequencies, indicated by circled numbers 1-9 and letter A on FIG. 6, were used as excitation frequencies. The conditions were experiment duration of 30 min, an oil/water emulsion with 1000 ppm oil concentration of approximately 5-micron SAE-30 oil droplets, a flow rate of 500 ml/min, and an applied power of 20 W.

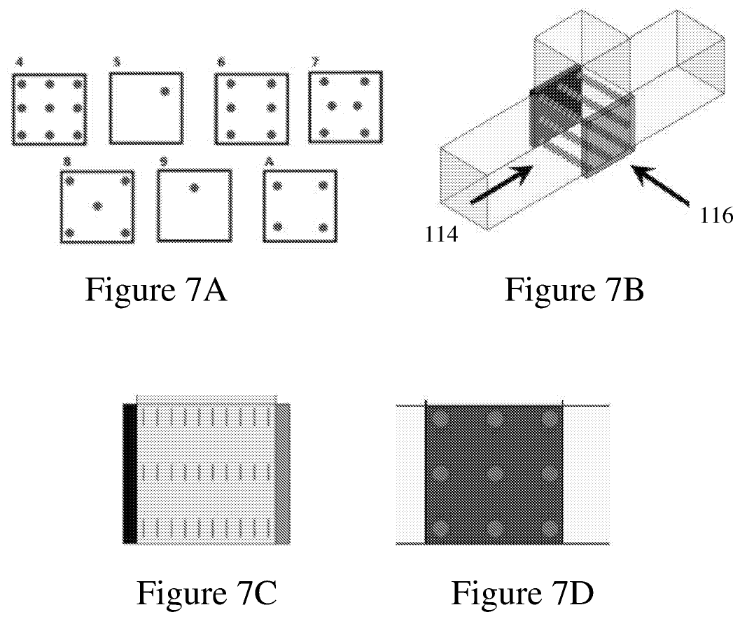

As the emulsion passed by the transducer, the trapping lines of oil droplets were observed and characterized. The characterization involved the observation and pattern of the number of trapping lines across the fluid channel, as shown in FIG. 7A, for seven of the ten resonance frequencies identified in FIG. 6.

FIG. 7B shows an isometric view of the system in which the trapping line locations are being determined. FIG. 7C is a view of the system as it appears when looking down the inlet, along arrow 114. FIG. 7D is a view of the system as it appears when looking directly at the transducer face, along arrow 116.

The effect of excitation frequency clearly determines the number of trapping lines, which vary from a single trapping line at the excitation frequency of acoustic resonance 5 and 9, to nine trapping lines for acoustic resonance frequency 4. At other excitation frequencies four or five trapping lines are observed. Different displacement profiles of the transducer can produce different (more) trapping lines in the acoustic waves, with more gradients in displacement profile generally creating higher trapping forces and more trapping lines. It is noted that although the different trapping line profiles shown in FIG. 7A were obtained at the frequencies shown in FIG. 6, these trapping line profiles can also be obtained at different frequencies.

FIG. 7A shows the different crystal vibration modes possible by driving the crystal to vibrate at different fundamental frequencies of vibration. The 3D mode of vibration of the crystal is carried by the acoustic wave across the fluid in the chamber all the way to the reflector and back. The resulting multi-dimensional acoustic wave can be thought of as containing two components. The first component is a planar out-of-plane motion component (uniform displacement across crystal surface) of the crystal that generates a acoustic wave, and the second component is a displacement amplitude variation with peaks and valleys occurring in lateral directions across the crystal surface. Three-dimensional force gradients are generated by the acoustic wave. These three-dimensional force gradients result in lateral radiation forces that stop and trap the particles with respect to the flow by overcoming the viscous drag force. In addition, the lateral radiation forces are responsible for creating tightly packed clusters of particles. Therefore, particle separation and gravity-driven collection depends on generating a multi-dimensional acoustic wave that can overcome the particle drag force as the mixture flows through the acoustic wave. Multiple particle clusters are formed along trapping lines in the axial direction of the acoustic wave, as presented schematically in FIG. 7A.

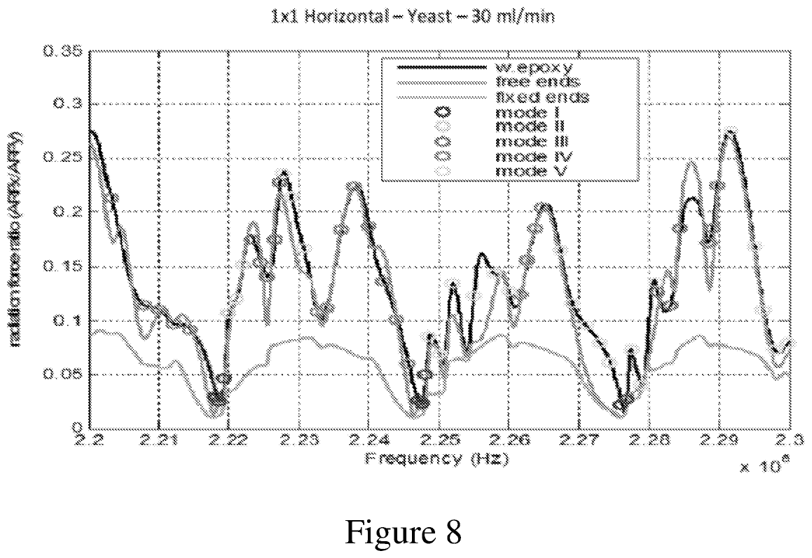

The piezoelectric crystals of the transducers described herein can be operated at various modes of response by changing the drive parameters, including frequency, for exciting the crystal. Each operation point has a theoretically infinite number of vibration modes superimposed, where one or more modes are dominant. In practice, multiple vibration modes are present at arbitrary operating points of the transducer, with some modes dominating at a given operating point. FIG. 8 presents COMSOL results for crystal vibration and lateral radiation forces on a typical particle size. The ratio of lateral to axial radiation force is plotted versus operating frequency. Points are labeled on the curve where a specific mode of vibration is dominant. Mode I represents the planar vibration mode of the crystal designed to generate a 2 MHz acoustic wave in a mixture. Mode III represents the 3×3 mode operation of a 1×1 crystal. These analytical results show that the 3×3 mode can be dominant with different levels of lateral radiation force. More specifically, operating the example system at a frequency of 2.283 MHz generates the lowest lateral force ratio of about 1.11 for a 3×3 mode. This operating point generates the largest cluster size and the best collection operation for the example system. Operating the devices and systems described herein at a frequency for a given configuration that produces a desired 3D mode with the lowest lateral force ratio is desirable to achieve the most efficient separation.

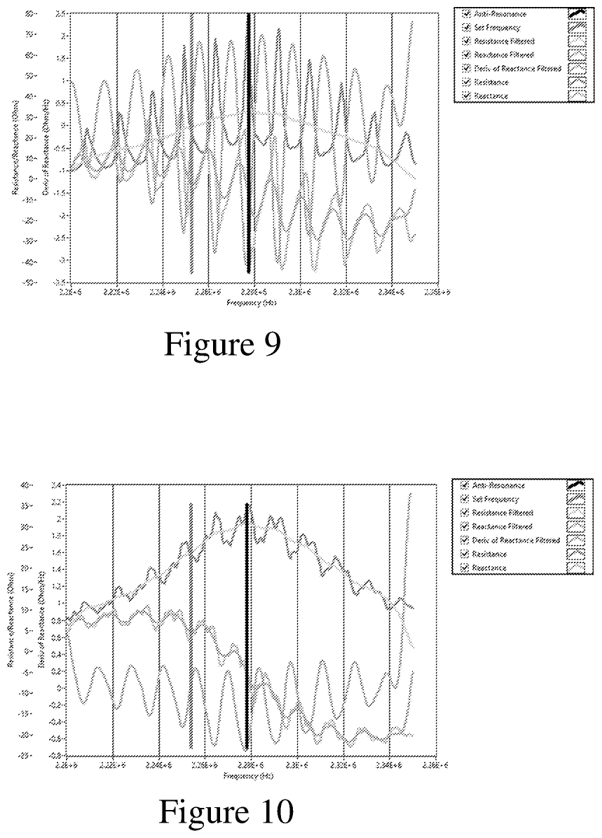

FIG. 9 illustrates a frequency scan for a slightly damped 1×3 piezoelectric transducer coupled to an acoustic cavity through which a fluid containing CHO (Chinese hamster ovary) cells was flowed. As illustrated, peak anti-resonance is located, and a minimum reactance two away from the anti-resonance is selected for a frequency setpoint. In the figure, anti-resonance is approximately 2.278 MHz, and the selected frequency setpoint is approximately 2.251 MHz.

FIG. 10 illustrates a frequency scan for a highly damped 2 MHz 1×3 transducer coupled to an acoustic chamber containing CHO. The peak anti-resonance is identified and the minimum reactance two away from the anti-resonance frequency is selected for an operating setpoint. Although a minimum reactance two away from the anti-resonance frequency is chosen as an operating setpoint, any reactance minima or index away from anti-resonance can be chosen for an operating setpoint.

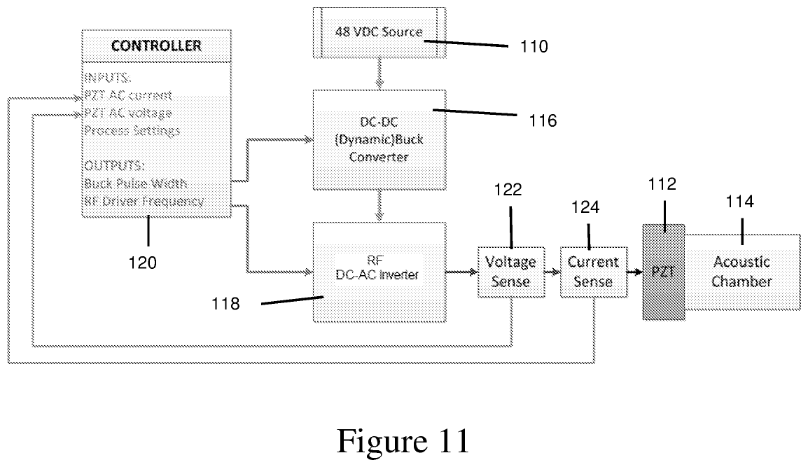

Referring to FIG. 11, a diagram of a control configuration for controlling an acoustic transducer 112 coupled to an acoustic chamber 114 is illustrated. Acoustic transducer 112 is driven by an RF power driver composed of DC source 110, DC-DC converter 116 and RF DC-AC inverter 118. The output drive signal provided by inverter 118 is inspected or sensed to obtain voltage sense 122 and current sense 124, which are fed back to a controller 120. Controller 120 provides control signals to converter 116 and inverter 118 to modulate the drive signal provided to the acoustic transducer 112.

The signal provided by controller 120 to converter 116 is a pulse width measure, which determines the duty cycle of the switching signals in converter 116. The duty cycle determines the output of converter 116, which is applied to a filter (not shown) to produce a DC signal, which is applied to inverter 118. For example, the greater the duty cycle, the higher the output that is generated by converter 116 and subsequent DC signal produced by the filter couple to the output of converter 116. Controller 120 also provides control signals to inverter 118 that determine the frequency of operation of inverter 118. The control signals provided to inverter 118 may be switching signals, for switching switches in inverter 118. Alternately, or in addition, controller 120 can provide a control signal to inverter 118 that is used to indicate a desired switching frequency, and circuitry internal to inverter 118 interprets the control signal and switches the internal switches in accordance with the interpreted control signal.

Voltage sense 122 and current sense 124 produce signals that are provided to controller 120 as feedback signals to control the drive signal provided to acoustic transducer 112. Controller 120 performs operations and calculations on the signals provided by voltage sense 122 and current sense 124, for example, to obtain a power measure, P=V*I, or to obtain a phase angle, θ=arctan (X/R).

Controller 120 is provisioned with a control scheme that accepts process settings, such as power output, range of frequency operation, or other user selectable parameters, and provides control signals to converter 116 and inverter 118 based on the process settings and the feedback values. For example, as described above, controller 120 can sequence through a number of frequencies in a range of frequencies that are provided to inverter 118 to scan through the frequency range and determine the characteristics of transducer 112 or transducer 112 in combination with acoustic chamber 114, which may be under load. The results of the frequency scan in terms of voltage and current obtained from the voltage sense 122 and current sense 124, respectively, are used to identify characteristics of the impedance curves for the components or the system, such as is illustrated in FIGS. 9 and 10. The frequency scan can be implemented to occur at set up, and/or at intervals during operation of the illustrated system. During steady-state operation, the frequency scanned can be conducted to identify desired setpoints for operation, such as power or frequency, based on user settings and feedback values. The control scheme implemented by controller 120 is thus dynamic, and responds to changing conditions in the system, such as may be encountered with frequency drift, temperature change, load changes and any other system parameter changes. The dynamic nature of the control scheme permits the controller to respond to or compensate for nonlinearities, such as may be encountered as components age or lose tolerance. Accordingly, the control scheme is adaptive and can accommodate system changes.

Some examples of system operation include driving acoustic transducer 112 to produce a multidimensional acoustic wave in the acoustic chamber 114. A 3D acoustic wave is stimulated by driving acoustic transducer 112, which may be implemented as a piezoelectric crystal, sometimes referred to herein as a PZT, near its anti-resonance frequency. Cavity resonances modulate the impedance profile of the PZT as well as affect its resonance modes. Under the influence of the 3D acoustic field, suspended particles in the liquid medium in the acoustic cavity 114 are forced into agglomerated sheets and then into strings of ‘beads’ of agglomerated material. Once particle concentrations reach a critical size, gravitational forces take over and the agglomerated material drops out of the acoustic field and to the bottom of the chamber. The changing concentrations of agglomerated material as well as the dropping out of that material affects the cavity's resonances which in turn change the acoustic loading on the PZT and its corresponding electrical impedance. The changing dynamics of the collected material detunes the cavity and PZT reducing the effects of the 3D wave in clarifying the medium. Additionally, changes in the medium and cavity temperature also detune the cavity so that clarification is reduced. To track the resonance changes occurring in the cavity, a control technique is used to follow changes in the PZT's electrical characteristics.

A strong 3D acoustic field can be generated by driving the PZT at a frequency where its input impedance is a complex (real and imaginary) quantity. However, cavity dynamics can cause that impedance value to change significantly in an erratic manner. The changes in impedance are due, at least in part, to changes in the load applied to the acoustic transducer 112 and/or acoustic chamber 114. As particles or secondary fluid is separated from a primary or host fluid, the loading on acoustic transducer 112 and/or acoustic chamber 114 changes, which in turn can influence the impedance of the acoustic transducer 112 and/or acoustic chamber 114.

To correct for detuning, controller 120 calculates the PZT impedance from the voltage and current sensed at the PZT using voltage sense 122 and current sense 124 and determines which way to change the operating frequency to compensate for the detuning. Since frequency changes affect power delivered to the chamber, the controller also determines how to adjust the output voltage of (dynamic) buck converter 116 to maintain the desired amount of power output from RF DC-AC inverter 118 and into the acoustic transducer 112 and/or acoustic chamber 114.

Buck converter 116 is an electronically adjustable DC-DC power supply and is the power source for inverter 118. RF DC-AC inverter 118 converts the DC voltage out of converter 116 back to a high-frequency, AC signal to drive the PZT. The dynamics in the chamber occur at rates corresponding to frequencies in the low audio band. Consequently, the converter 116, controller 120, and DC-AC inverter 118 are capable of working at rates faster than the low audio band to permit controller 120 to track chamber dynamics and keep the system in tune.

Controller 120 can simultaneously change the frequency of DC-AC inverter 118 and the DC voltage coming out of buck converter 116 to track cavity dynamics in real time. The control bandwidth of the system is a function of the RF bandwidth of inverter 118, the cutoff frequency of the filtering system of buck converter 116 and the RF bandwidth of acoustic transducer 112.

Controller 120 can be implemented as a DSP (digital signal processor) control, or as an FPGA (field programmable gate array) control, as examples. Controller 120 may be implemented with two channels, to permit parallel processing, for example to analyze real and/or reactive impedance, voltage, current and power.

The acoustic dynamics of the cavity affects the electrical characteristics of the PZT which affects the voltage and current drawn the PZT. The sensed PZT voltage and current is processed by the controller to compute the real-time power consumed by the PZT as well as its instantaneous impedance (affected by acoustic dynamics). Based on user set points the controller adjusts, in real-time, the DC power supplied to inverter 118 and the frequency at which inverter 118 is operated to track cavity dynamics and maintain user set points. An LCL network is used to match the output impedance of inverter t 118 to increase power transfer efficiency.

Controller 120 samples sensor signals fast enough to detect changes in cavity performance (via changes in PZT impedance) in real time. For example, controller 120 may sample the feedback values from the voltage sense 122 and current sense 124 at one hundred million samples per second. Signal processing techniques are implemented to permit a wide dynamic range for system operation to accommodate wide variations in cavity dynamics and applications. Converter 116 can be configured to have a fast response time to follow the signal commands coming from controller 120. Inverter 118 can drive a wide range of loads that demand varying amounts of real and reactive power that change over time. The electronics package used to implement the system illustrated in FIG. 11 may be configured to meet or exceed UL and CE requirements for electromagnetic interference (EMI).

Controller 120 may be implemented with very-high-speed parallel digital-signal-processing loops using RTL (Register Transfer Level) which is realized in actual digital electronic circuits inside a field-programmable-gate-array (FPGA). Two high speed digital proportional integral (PI) loops adjust the frequency and amplitude control signals generated by controller 120 to track power and reactance. The voltage and current sense is used to sense the voltage and current at the transducer. The FPGA can be operated with a clocking signal of 100 MHz. The clocking speed contributes to obtaining fast enough sampling to monitor and adapt to conditions of the PZT in real-time. In addition, the structure of the FPGA permits each gate component to have a propagation delay commensurate with the clocking speed. The propagation delay for each gate component can be less than one cycle, or 10 ns with a clocking speed of 100 MHz.

Parallel and sequential operations for calculating control signals can be implemented by controller 120 to calculate the following parameters.

VRMS=sqrt(V1²+V2²+ . . . +Vn²)

IRMS=sqrt(I1²+I2²+ . . . +In²)

Real Power (P=V-Inst.×I-Inst Integrated over N Cycles)

Apparent Power (S=VRMS×IRMS)

Controller 120 may be configured to calculate reactive power and bipolar phase angle by decomposing sensed voltage and current into in-phase and quadrature-phase components. In-phase and quadrature-phase demodulation of the voltage and current can be implemented to obtain a four-quadrant phase, reactive power and reactance. The calculations for reactive power and phase angle can be simplified using the in-phase and quadrature-phase components.

VPhase Angle=Arctan(QV/IV)

IPhase Angle=Arctan(QI/II)

Phase Angle=VPhase−Iphase

Reactive Power=(Q=Apparent Power×Sine(Phase Angle)

The above calculations and results can be used to determine the state of the acoustic system, including loading in the acoustic chamber. For example, when the acoustic chamber has a higher than average amount of material, or when the acoustic wave is holding more material, the system is more heavily loaded and the reactance minimum of the system can shift in frequency accordingly. When the acoustic chamber has less material, or when the acoustic wave is holding less material, the system loading is less, and the reactance minimum shifts in frequency accordingly. For continuous operation, it is desirable to have steady state operation that accommodates or compensates for changes in loading. The tracking of the minimum reactance described herein can achieve high performance in continuous operation. However, the initial conditions of the acoustic system can be challenging to accommodate with a steady state control.

According to an example implantation, controller 120 may implement a startup procedure to initialize the acoustic system and prepare it for operation. The procedure may begin with a fault detection query and/or reset to determine if components and conditions are within desired operating bounds. Fault detection can include detection of open circuits, short circuits, over temperature, excessive power, and any other condition that may be undesirable or pose a danger to or problem for operating the acoustic system.

The process assumes that the system has been configured for normal operation to get a data set of Zsys(f) that has the proper information to allow R/X tracking. Such a data set can be achieved by scanning through a number of discrete frequencies in a frequency range, and measuring system parameters, such as voltage and current, at each discrete frequency. Since a PZT's frequency characteristics change as a function of PZT loading, a frequency scan is implemented under typical operating conditions to improve the accuracy of the information. Better system performance is obtained by not operating at the PZT's anti-resonant frequency, f2. Therefore, the startup procedure implements a process that is relative to f2 instead of f1 to identify and avoid the Xmin closest to f2. For example, f2 is used as a starting point for the computations in the startup procedure. The startup procedure tracks current system conditions using the PZT model calibrated against the initial frequency sweep conditions. The model may be maintained and not recalibrated during normal system operation or may be recalibrated at certain intervals that may be periodic or random or related to system operational status or parameters. The model may be recalibrated upon the occurrence of certain events. For example, if the system drifts too far outside of acceptable performance ranges, the model may be recalibrated. The ranges of acceptable performance may be determined from a number of parameters or combinations of parameters.

According to another example implementation, a startup procedure initializes and prepares the acoustic system for operation. The procedure stabilizes the acoustic chamber in an initial state prior to continuous operation. The procedure uses controller 120 to implement a control scheme that begins with a frequency sweep to determine system performance parameters at discrete frequencies within the frequency sweep range. The control scheme may accept inputs of a start frequency, a frequency step size and number of steps, which defines the frequency sweep range. Controller 120 provides control signals to modulate the frequency applied to acoustic transducer 112, and the voltage and current of the crystal are measured using voltage sense 122 and current sense 124. The control scheme of controller 120 may repeat the frequency sweep a number of times to determine the system characteristics, for example, reactance, with a relatively high level of assurance.

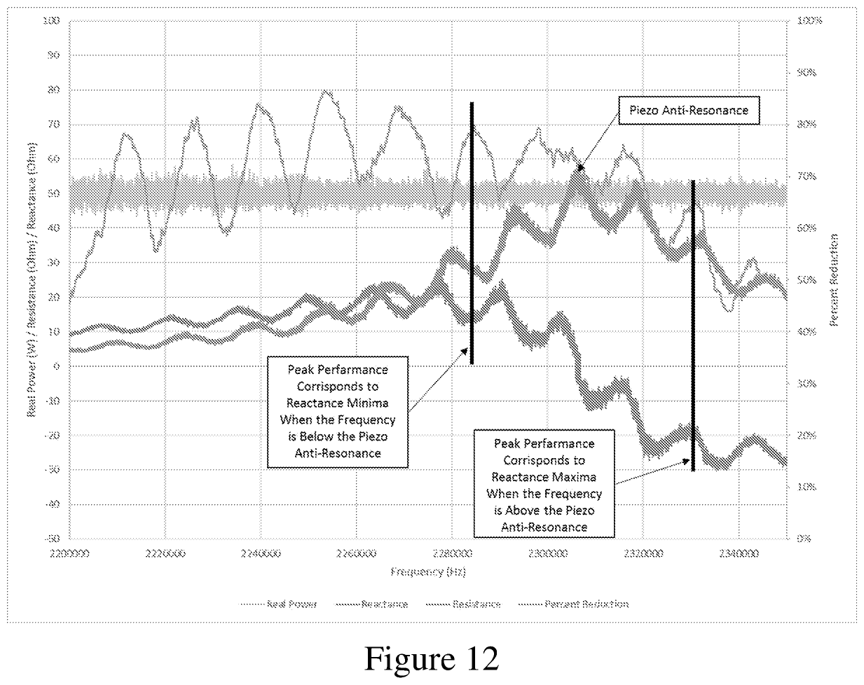

Through experimental testing of the large scale acoustic filtration system, it has been determined that the 1 MHz and 2 MHz 1×3 transducer may have an optimal efficiency when operating at the minimum reactance points at frequencies below the transducer anti-resonances, as well as operating at the maximum reactance points above the anti-resonance of the transducer, as illustrated in FIG. 12. The technique described herein provides an automated startup method to prepare the resonance chamber for operation and set the frequency of the RF drive to the transducer, so it is operating at a minimum reactance point below the anti-resonance or a maximum reactance above the anti-resonance. According to a feature, the technique intermittently or continuously determines at what frequency the minimum reactance is located, and sets the frequency of the drive for the acoustic transducer 112 to that frequency. The technique can be used to set and adjust the frequency of inverter 118 to operate the RF drive.

TABLE 1
Functions and Variable Inputs and Outputs

| Name | Type | Description |
|---|---|---|
| Scan | Function | Steps through a range of frequencies and |
| Function | | captures Resistance and Reactance data |
| | | from the Voltage and Current |
| | | measurements of the RF drive. |
| | | Inputs: |
| | | Range (+−50 kHz around anti-res) |
| | | Step Size (500 Hz) |
| | | Step Interval (1 ms) |
| | | Output: Array of Frequency, R, and X |
| Estimated | Input | Expected number of resonances over the |
| Number of | Double | full scan range |
| Resonances | | |
| Number of | Input | If negative the method will pick the |
| Reactance | Signed | frequency of that many minima below the |
| Minima/Maxima | Integer | anti-resonance. If positive the method will |
| from Anti- | | pick the frequency of that many maxima |
| Resonance | | above the anti-resonance |
| Frequency | Output | The frequency that the method picks to set |
| to Set | Double | the RF drive |
| Wait Time | Input | Specifies the amount of time between |
| | Double | scans |

The method begins by running a sweep of frequencies and collecting resistance and reactance data for each frequency step. The resistance and reactance data is extrapolated from the voltage and current measurements of the RF drive. The sweep range is specified by the user, but is targeted to be 50 kHz above and 50 kHz below the anti-resonance of the transducer. The step size and step interval are also variables that can be altered. When the sweep is complete it outputs the frequency, resistance, and reactance at each step.

The data from the sweep is then filtered utilizing a zero-phase low pass Butterworth filter. The reactance enters a loop where the low cutoff frequency of the filter is constantly increased, until the number of peaks of the filtered data, equals the number of estimated peaks. This number of estimated peaks is entered by the user. The resistance data is filtered using a zero-phase low-pass Butterworth filter, however the low cutoff frequency is increased until there is one peak. The peak value of the filtered resistance data is interpreted as the anti-resonance of the transducer.

The derivative of the filtered reactance data is calculated and is used to find all the maximum or minimum points of the reactance curve. If the number of reactance minima/maxima from the anti-resonance data input is negative the method will look for the minimum reactance points below the anti-resonance. The method does this by identifying the negative to positive zero crossings, in other words, the upward slope zero crossings of the derivative of the filtered reactance curve. If this number is positive the method will look for the positive to negative zero crossings above the anti-resonance, which are the maximum points of the reactance curve. The absolute value of the number of reactance minima/maxima from the anti-resonance data input is the number of minimum or maximum points from the anti-resonance. The index of this point is used to determine the frequency to set the RF drive.

The RF drive is set and the method waits for a designated amount of time set by the user. Once this time period has elapsed the method then scans and start the sequence over again. Sample data of both slightly and highly damped data can be seen in FIGS. 9 and 10. In both these examples the method was selected to pick two minimum reactance points below the anti-resonance. The set frequency is indicated by the red line in FIGS. 9 and 10. It can be seen that this line falls on the negative to positive zero crossing of the derivative of the filtered reactance data curve, and at the local minimum of the filtered reactance data curve.

A number of reactance minimums can be identified as a result of analysis of the data obtained in the frequency sweep. The control technique can be provided with an input that specifies a certain frequency range where a desired reactance minimum is located, as well as being provided with a resistance slope (+/−) that can be used for tracking a desired point of operation based on resistance tracking that corresponds to a desired minimum reactance. The resistance slope may be constant near the minimum reactance, which may provide a useful parameter for use with a tracking technique. By tracking resistance at a desired frequency, a robust control can be attained for operating at a minimum reactance point.

The control technique may take the derivative of the resistance/reactance values to locate zero slope derivatives, which are indicative of maximums and minimums. A proportional-integral-differential (PID) controller loop may be used to track the resistance to obtain a frequency setpoint at which a desired minimum reactance occurs. In some implementations, the control may be a proportional-integral (PI) loop. With the FPGA operating at 100 MHz, adjustments or frequency corrections can be made every 10 ns to compensate for changes in the tracked resistance. This type of control can be very accurate and implemented in real-time to manage control of the PZT in the presence of a number of changing variables, including reactance, load and temperature, for examples. The control technique can be provided with an error limit for the frequency of the reactance minimum or frequency setpoint, to permit the control to adjust the output of inverter 118 to maintain the frequency within the error limit.

A fluid mixture, such as a mixture of fluid and particulates, may be flowed through the acoustic chamber to be separated. The fluid mixture flow may be provided via a fluid pump, which may impose perturbations on the fluid, as well as the PZT and chamber. The perturbations can create a significant fluctuation in sensed voltage and current amplitudes, indicating that the effective impedance of the chamber fluctuates with pump perturbations. However, owing to the speed of the control technique, the fluctuations can be almost completely canceled out by the control method. For example, the perturbations can be identified in the feedback data from the PZT and can be compensated for in the control output from the controller. The feedback data, for example the sensed voltage and current, may be used to track the overall acoustic chamber pressure. As the characteristics of the transducer and/or acoustic chamber change over time and with various environmental parameters, such as pressure or temperature, the changes can be sensed and the control technique can compensate for the changes to continue to operate the transducer and acoustic chamber at a desired setpoint. Thus, a desired setpoint for operation can be maintained with very high accuracy and precision, which can lead to optimized efficiency for operation of the system.

The FPGA may be implemented as a standalone module and maybe coupled with a class-D driver. Each module may be provided with a hardcoded address so that it can be identified when connected to a system. The module can be configured to be hot-swappable, so that continuous operation of the system is permitted. The module may be calibrated to a particular system and a transducer, or may be configured to perform a calibration at particular points, such as upon initialization. The module may include long-term memory, such as an EEPROM, to permit storage of time in operation, health, error logs and other information associated with operation of the module. The module is configured to accept updates, so that new control techniques can be implemented with the same equipment, for example.

Controller 120 can implement a method for controlling an acoustic transducer. The method uses a low voltage output during a frequency sweep that drives the acoustic transducer over a range of frequencies. Feedback from the acoustic transducer is used to determine the resistance and reactance response of the transducer over the range of frequencies at the low voltage output. Once the data for the transducer responses collected, the frequency at which the minimum reactance occurs below anti-resonance is identified. The resistance at the minimum reactance is identified and the frequency setpoint is set to establish operation at this resistance. A real power setpoint for the frequency setpoint is established, which may be based on user input. The establishment of the operating setpoints, the method causes the power control signals to be output for the linear amplifier or the converter-inverter power supply.

The method performs a loop in which voltage and current are measured at the acoustic transducer, real power and resistance are calculated and provided to a proportional-integral (PI) controller. The output of the PI controller is used to adjust the amplitude and frequency of the signal supplied to the transducer. The loop is repeated, resulting in the amplitude of the power provided to the transducer being controlled and tracked, and the frequency of the power provided to the transducer being controlled and tracked. The loop permits the controller to dynamically adjust to changes in the system, including changes related to loading of the transducer and/or the transducer/acoustic cavity combination or changes related to temperature, as examples.

Controller 120 can implement a method for processing information to implement a transducer control. The method uses desired operating points for real power and a minimum reactance, which may be obtained from user input. Data is received from the transducer, including drive voltage and drive current. The data received from the transducer is conditioned to improve the quality of the information and calculations derived there from. For example, the data representing drive voltage and drive current is deskewed, provided with an offset and scaled for use with subsequent calculations. The condition data is used to calculate real power, resistance and reactance of the transducer. These parameters are compared to operating points received in the method, and a PI controller is used to generate a signal that can adjust the real power and frequency of the drive signal provided to the transducer. Note that the conditioned feedback parameters can be used to generate an error signal in conjunction with the desired operating point information, with the error signal being provided to an amplifier that adjusts the signal provided to the power supply, whether linear amplifier or converter-inverter combination.

The graph in FIG. 12 shows reactance minima and maxima that can be used for operating points in the acoustic system. Real power is relatively constant. In this example, the input real power and acoustic real power are fairly well matched, indicating efficient transfer of power. In practice, a trade of choices can be made between operating the transducer to obtain highly efficient separation in the acoustic chamber, implying a minimum reactance point, and obtaining efficient power transfer into the chamber. For a given material being separated and a given transducer, filter components can be selected with a resonance frequency to obtain efficient power transfer into the acoustic cavity, improving overall system efficiency.

Turbidity performance is used to measure efficiency of separation in the acoustic system. The acoustic transducer is operated at a reactance minimum, and at a point that represents multimode operation, which can produce axial and lateral forces on particles in the fluid through which the acoustic wave passes. Thus, providing a control technique for operating the acoustic transducer at a reactance minimum can attain desired performance. The desired performance can be attained even at zero phase when operating in multimode, unlike the zero-phase planar wave operation. This result shows the significant advantages in terms of performance for multimode operation at minimum reactances. These performance benefits are not obtained with zero or planar wave mode of operation for the transducer.

Improved performance of a three-dimensional field (multi-dimensional) acoustic wave system can be achieved by operating at a frequency where the reactive component of the input impedance of the PZT-cavity system is at a minimum. FIG. 12 illustrates where those conditions exist in a 2 MHz system. FIG. 12 shows that there are multiple resonances over the working frequency band of a PZT-cavity system and that response characteristics have a quasi-periodic nature. The periodic nature of the resonant cavity is influenced by the non-periodic characteristics of the PZT. The PZT caused distortions of the cavity operation make it difficult to establish an automatic control process for the PZT-cavity system.

Cavity resistance, Rc, (that acoustic component which does useful work) is essentially periodic. Its maxima coincide with maximum transduction efficiency. These maxima also align with the reactance minima of the PZT's input impedance (more so the further away from anti-resonance one goes). However, the value of one particular reactive minimum is different from other reactive minima. The resistive and reactive curves, from cavity influences, ‘ride’ the Rpzt and Xpzt curves of the PZT. These curves change under dynamic conditions. Temperature changes in the cavity cause the resonant curves to shift laterally in frequency. Such a shift causes a particular minimum reactance, Xmin, to change value because it is ‘sliding’ along the Xpzt curve. The Xmin value relative to Xpzt level has little or no change (due to secondary PZT effects). If there are no temperature changes but there is a change in the damping (increase or decrease in energy absorption) in the cavity there will again be a change in the value of a particular Xmin. The change takes the form of an increasing or decreasing peak-to-peak value in X relative to Xpzt. That change in value due to damping changes can easily be confused for changes in temperature. Thus, devising a control scheme that automatically tracks a particular Xmin value should be designed to distinguish between damping changes which do not move Xmin and thermal drift changes which do move Xmin. The following presents a procedure and associated algorithms for finding and tracking a particular Xmin under dynamic conditions.

The method used to minimize the effects of Rpzt and Xpzt in R and X is to make use of a mathematical model which closely represents Rpzt and Xpzt over the frequency band of interest. That model's response shape is subtracted from the system's response shape to minimize the PZT's distortion effects on cavity dynamics. The model's impedance function is given as

$${Z}_{o} ( f ) = \frac{1}{j 2 π {fC}_{o}} ⌈ \frac{Q ( {f}_{1}^{2} - {f}^{2} ) + {jff}_{1}}{Q ( {f}_{2}^{2} - {f}^{2} ) + {jff}_{1}} ⌉ = {R}_{o} ( f ) \pm {jX}_{o} ( f ) \qquad ( 1 )$$

where
f=evaluation frequency in Hz
Cₒ=native capacitance of the PZT structure
f₁=resonant frequency of the PZT
f₂=anti-resonant frequency of the PZT
Q=PZT quality factor associated with energy absorbed by the structure and its connected load
j=imaginary operator (complex number notation)
Rₒ(f)=resistive or real part of Zₒ(f)

$${R}_{o} ( f ) = \frac{1}{2 π {fC}_{o}} \frac{{Qf}_{1} ( {f}_{2}^{2} - {f}_{1}^{2} ) f}{{{Q}^{2} ( {f}_{2}^{2} - {f}^{2} )}^{2} + {f}_{1}^{2} {f}^{2}} \qquad ( 1 a )$$

Xₒ(f)=reactive or imaginary part of Zₒ(f)

$${X}_{o} ( f ) = \frac{1}{2 π {fC}_{o}} \frac{{Q}^{2} ( {f}_{1}^{2} - {f}^{2} ) ( {f}_{2}^{2} - {f}^{2} ) + {f}_{1}^{2} {f}^{2}}{{{Q}^{2} ( {f}_{2}^{2} - {f}^{2} )}^{2} + {f}_{1}^{2} {f}^{2}} \qquad ( 1 b )$$

The model's response, subtracted from the system's response corrects distortions introduced by the PZT. The proper choice of Q (the parameter that controls the shape of the model's frequency response) can essentially cancel the response distortions introduced by the PZT. The effect of canceling the PZT distortions produces reactance responses that are more symmetric about zero. Such symmetry makes it easier to detect changes in drift compared to changes in damping. A drift change moves a particular Xmin in frequency with little or no in change its value since it is no longer tracking the reactance curve of the PZT. A change in damping will change the magnitude value of a particular Xmin but there will be no change in the frequency direction. Thus, tracking Xmin with the model compensation involves tracking thermal drift. There is still a frequency component of the PZT that distorts cavity effects. The PZT's ability to respond to cavity dynamics decreases as one moves away from the PZT's anti-resonant frequency. Extrema in either R or X do not hit the same value across the frequency band but taper off in the form of a bell shaped curve. This distortion provides a cross coupling between damping and drift but not to the same extent as when an uncorrected response is used. A method of reducing the effects of this cross coupling are presented later in this paper.

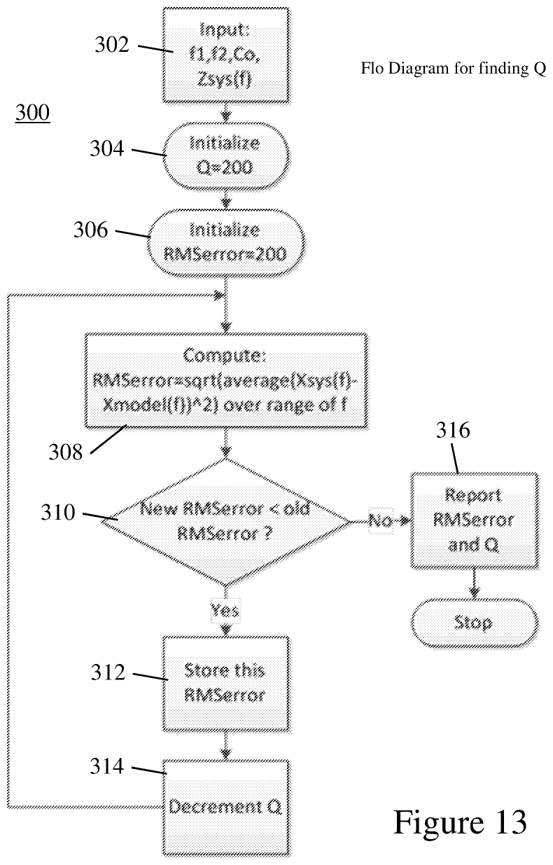

Determining the proper value of Q is found through the use of an iterative process which is illustrated in flowchart 300 in FIG. 13. The values of the PZT's resonant and anti-resonant frequencies, f1 and f2, the device's native capacitance, Co, and a data set of the PZT-cavity input impedance, Zsys, over a frequency range that includes f1 and f2 are input into the process as shown in block 304. Blocks 304 and 306 show the process being initialized with values of Q and RMS error that are much greater than anything that might be encountered in an actual system. For each iteration of the process, an RMS error is computed and compared to its previous value as shown in blocks 308 and 310. If the new error is smaller than the old error (the “Yes” branch) then the new replaces the old in block 312 and the old value of Q is decremented a fixed amount and replaced by its new value in block 314. The process repeats until a decrease in Q causes an increase in error, as determined in block 310. At that point, (the “No” branch) the process is stopped and the final value of Q is stored as shown in block 316 to be used in subsequent signal processing to track dynamic changes in the acoustic cavity. The RMS error calculation is given as

$$RMS error = \sqrt{\frac{1}{N} {\sum }_{n = 0}^{{N}_{s}} {[ {X}_{sys} ( {f}_{n} ) - {X}_{o} ( {f}_{n} ) ]}^{2}} \qquad ( 2 )$$

where
Nₛ=number of samples taken in doing a frequency scan of the system
fₙ=discrete frequency values used in the frequency scan
X_{sys}(fₙ)=sample value of the system's reactance found at frequency fₙ
Xₒ(fₙ)=sample value of model's reactance computed at fₙ

The error calculation gives a measure of how different two functions are from each other on a pointwise basis. In the present example, the error may not go to zero since the system function includes the periodic changes in impedance due to cavity effects whereas the PZT model does not contain those cavity effects. The objective of this process is to remove PZT effects and keep cavity effects.

Since there are multiple cavity resonances, with corresponding reactance minima, across the frequency band of interest (e.g. f1 to f2), a method may be devised that permits automatic minima locating to be implemented. One possible example method is described next. The observed, periodic cavity resonances detected in a frequency sweep of the PZT-cavity system are related to the acoustic path length of the cavity. The longer the path length, the more resonances will be observed over the given sweep interval. With the knowledge of the number of resonances contained in a given frequency sweep, the sweep band can be partitioned into segments that can be analyzed to find a minimum reactance and its frequency location in each segment. The resonant frequency, fc, of a given acoustic cavity is given as

$${f}_{c} = \frac{{v}_{c}}{2 {L}_{c}} \qquad ( 3 )$$

where
f_{c}=resonant frequency in Hz
v_{c}=velocity of sound within the cavity medium in meters/sec
L_{c}=cavity length in meters

The number of resonances, Nᵣ, within the PZT frequency band of f1 to f2 is given as

$${N}_{r} = \overline{( \frac{{f}_{2} - {f}_{1}}{{f}_{c}} )} \qquad ( 4 )$$

where Nᵣ is rounded to the nearest whole number since a fractional resonance is not real.

Knowing the number of data samples, Nₛ, taken to do a frequency sweep of the system, we can determine the number of data samples per resonance interval, N_{s/r}, there must be.

$${N}_{s \text{/} r} = \overline{( \frac{{N}_{s}}{{N}_{r}} )} \qquad ( 5 )$$

where N_{s/r} is also rounded to the nearest integer since a fractional sample is not real.

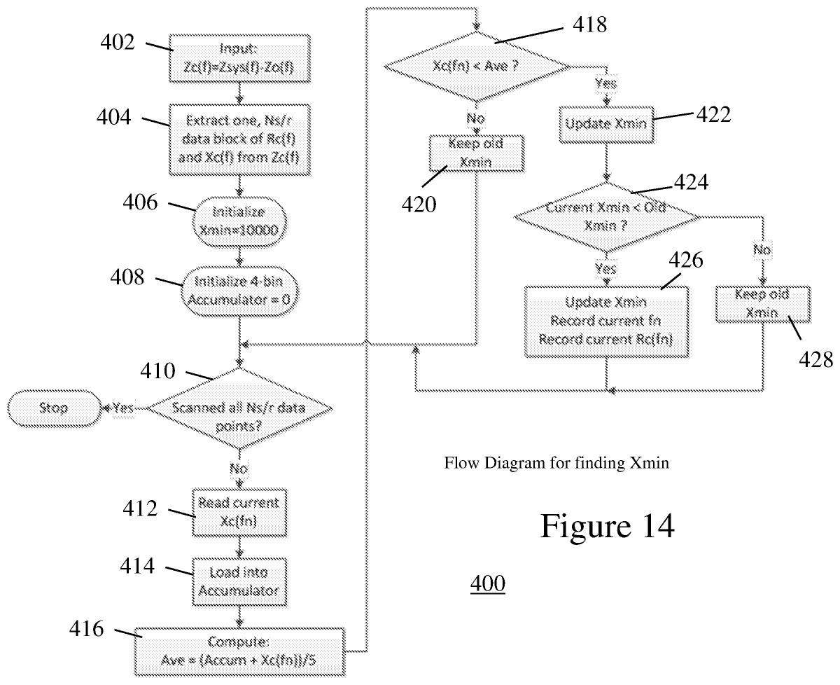

Determining the location and value of an Xmin in each resonance interval is performed in an iterative manner, as illustrated in flowchart 400 in FIG. 14. The process assumes that a frequency sweep of the system has been done and that the data has been filtered through a PZT correction process, e.g., as discussed above, the Q adjusted impedance of the PZT model has been subtracted from the system's impedance data to produce a corrected impedance, Zc, data set, as shown in block 402. A reactance minima range of frequencies and associated values is identified, as shown in block 404. The value for Xmin is initialized in block 406 and the accumulator for comparing Xc is initialized in block 408. The sample values are iteratively reviewed to determine if a new Xmin is found, as shown in block 410. Xmin is found by comparing the current value of Xc with the average value of four previous values plus the current Xc. If the current value is less than the average then the current value becomes the latest Xmin, as shown in blocks 412, 414, 416, 418, 420 and 422. As long as the updated value of Xmin continues to decrease, the new value for Xmin continues to be stored, as shown in blocks 424, 426 and 428. If the value of Xmin increases from the previous value, then the stored value for Xmin is not updated and the latest minimum value is stored or maintained. It is possible to have more than one local minimum in an Xc data set. Therefore, the current Xmin is compared against any past or ‘global’ Xmin in blocks 424, 426 and 428. If the current value is smaller than the last value then the current Xmin replaces the global Xmin and the associated frequency and Rc values are stored for future use. The reason for using a running average of Xc values in blocks 412, 414 and 416 is to reduce the effects of noise in the data. For conditions with more noise the number of Xc values used in the averaging process can be increased to compensate. To find Xmin in any resonance interval the index pointer into the Zc(f) data set is adjusted in multiples of Ns/r with the multiple anywhere between 0 and Nr.

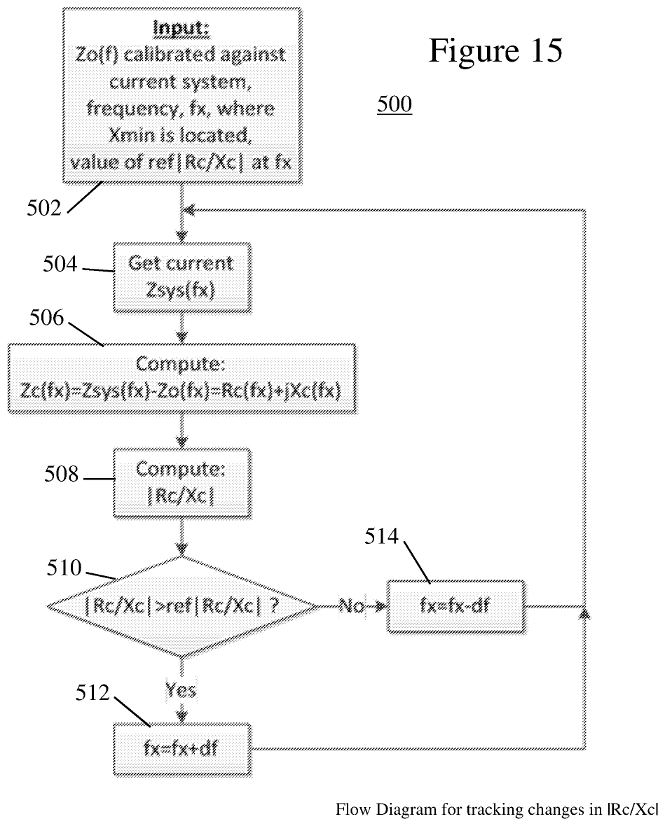

The determined values of Xmin, the frequency it is located at, and the associated Rc at that same frequency, can be used for an automatic tracking mode. Tracking the value of Xmin by itself may be complex and challenging. If the cavity resonance changes, detecting the direction of drift may be difficult because Xmin is located in a ‘value valley’ and may exhibit the same change in value from either direction of drift in the frequency direction. If cavity damping changes then the magnitude of Xmin changes, which may be difficult to differentiate from a drift change. If one tracks on the value of Rc located at the same frequency as Xmin, tracking conditions are improved a bit because Rc has a negative slope within the frequency range that brackets the location of Xmin. If the cavity drifts down in frequency the value of Rc will decrease and vice versa if the drift is up in frequency. Drift, as well as drift direction, is readily discernable. However, changes in damping will also change Rc so there is still coupling between damping and drift. This coupling effect is reduced by tracking on the ratio of Rc/Xc. The ratio method allows drift detection from changes in Rc and reduces the effects of damping changes since Rc and Xc tend to scale together with changes in damping. There are a number of ways of tracking a target value, FIG. 15 illustrates one such method.

The principle of operation illustrated in flowchart 500 in FIG. 15 is a simple bang-bang controller (e.g. how a basic thermostat works). The inputs tot the process are shown in block 502. The system impedances are determined as shown in block 506. The frequency, fx, that is used for driving the PZT-cavity system is incremented or decremented a fixed amount, df, based on whether the current |Rc/Xc| sample value, computed in block 508, is above or below the reference value, as shown in blocks 510, 512 and 514. This form of control introduces jitter into the drive frequency since the frequency change is always the same no matter how small or large the difference is between the current sample value and the reference. A smoother control may be implemented with a proportional controller that makes changes proportional to the difference between a current sample and the target reference value. As the difference becomes smaller the frequency change gets smaller, and vice-versa, thereby reducing jitter.

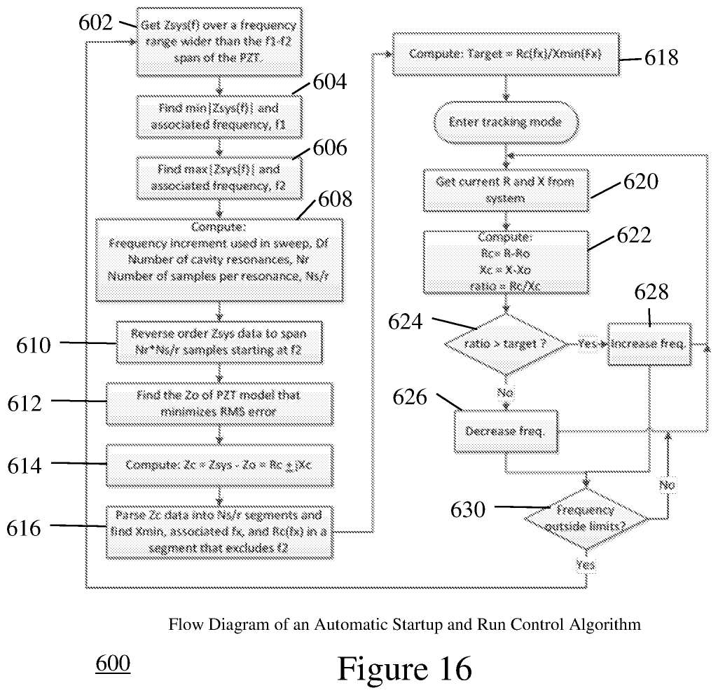

The above processes can be integrated into a control algorithm that provides an automatic startup and run procedure for a 3D acoustic wave system. The flow diagram 600 for this algorithm is shown in FIG. 16. A frequency scan over a wide range of frequencies, or a global frequency scan, is initiated as shown in block 602. Minimum and maximum impedance absolute values and associated frequencies are determined, as shown in blocks 604 and 606. The parameters for the startup procedure are determined, including frequency increment, number of cavity resonances and number of samples per resonance, as shown in block 608. The frequency range and number of samples is set as shown in block 610. A compensation model that reduces RMS error is found as shown in block 612. The current impedance is calculated, as shown in block 614. An Xmin is located in the current impedance segment that was previously calculated, as shown in block 616. A target operating point is computed, as shown in block 618. System impedance is determined, as shown in block 620 and current resistance and reactance and their ratio is calculated, as shown in block 622. If the ratio is greater than the target the frequency is increased, as shown in blocks 624 and 628. If the ratio is not greater than the target, the frequency is decreased, as shown in blocks 624 and 626. A frequency range check is performed, as shown in block 630, and the process continues to iterate.

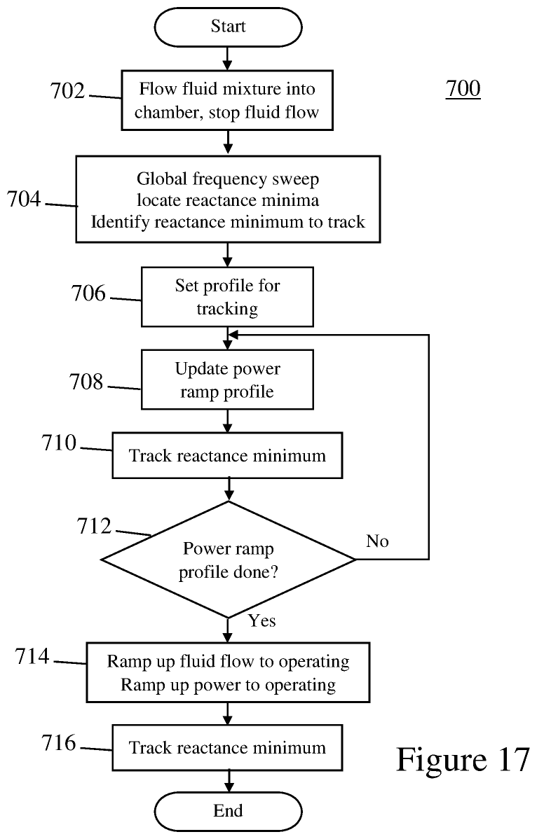

Another example of a startup procedure for the acoustic system is illustrated in flowchart 700 in FIG. 17. The process illustrated in flowchart 700 seeks to compensate the acoustic system during initialization in preparation for continuous operation. An operating frequency or range of frequencies is sought to be determined for use in continuous operation. However, if the acoustic system is immediately operated in continuous mode at the outset, a significant amount of material may not be captured by the acoustic wave, leading to initial performance degradation that can have a significant impact. The startup procedure is designed to obtain operating parameters for continuous operation, without significant loss of material that might otherwise be captured. In certain circumstances, the material can have a high cost or be the result of processing that utilizes significant resources, such that significant loss of material during startup is highly undesirable.

The startup procedure illustrated in flowchart 700 begins with flowing a fluid mixture into the acoustic chamber to fill the chamber, at which point the fluid flow is stopped, as shown in block 702. With the chamber filled with the fluid mixture, a global frequency sweep is conducted to locate reactance minima, as shown in block 704. A predetermined reactance minimum located in the frequency sweep may be identified for tracking, as also shown in block 704. The reactance minimum is at a frequency below anti-resonance for the acoustic system. The choice of the reactance minimum may depend on a number of factors, including the material type, acoustic system parameters, and other factors that can influence performance. The reactance minimum may be selected for the best performance to be obtained in the given system set up.

Block 706 shows the setting of a profile for tracking the selected reactance minimum. The profile may include a number of parameters for the tracking algorithm, including frequency step size, number of steps, frequency radius, step range in hertz, sweep radius and/or a time interval between steps. These and other parameters may be used in a profile or recipe for a given system set up. For example, a specific profile may be chosen for an acoustic chamber of a certain length, with an acoustic transducer operated at a certain frequency range, for processing a particular material with predetermined acoustic characteristics. The profile may include algorithm parameters for a breadth of frequency range that is treated with less sample granularity, which may represent a greater length of time for processing. The algorithm parameters may be set to increase sample granularity with smaller frequency ranges, which may ultimately speed the length of time for processing.

Once the reactance minimum is identified for tracking, and the tracking profile is established, the power applied to the acoustic transducer is cycled in a particular pattern, as shown in block 708. Patterns and that are used for the applied power may include ramping up power at a certain rate to a given level, dwelling at the level for a period of time, and ramping down power at a certain rate to a low power level. This pattern can be repeated a number of times in sequence while the frequency of the reactance minimum is monitored and used as an operating point for acoustic transducer operation. The frequency operating setpoint tracks the reactance minimum while the applied power is cycled in the particular pattern, as shown in block 710. The purpose of tracking the reactance minimum while cycling power is to cause material to settle or rise out of the acoustic wave in the acoustic chamber. The material leaving the acoustic wave does so gradually as power is cycled while the operating frequency tracks reactance minimum. With this technique, the loading in the acoustic wave is reduced, while the operating parameters for continuous operation, including reactance minimum, are determined. The material leaving the acoustic wave is not lost, and remains in the acoustic chamber in a clustered state. The cycling of the power during this startup phase reduces the potential rise in temperature that might otherwise be observed from the application of continuous full power.

Once the acoustic chamber is clarified of material via the cycling of the power, as shown in block 712, the acoustic system is ready for continuous operation. The fluid flow into the acoustic chamber is increased to a continuous operating range, as is the power applied to the acoustic transducer, as shown in block 714. The reactance minimum is continually tracked for use as a frequency operating setpoint as the acoustic system is brought into continuous operation, as shown in block 716.

Any type of reactance minimum tracking technique may be used in accordance with the startup procedure implementations discussed herein. For example, a global frequency scan can identify a number of reactance minima, one of which can be chosen as an operating point. The tracking algorithm can be based on measuring resistance, reactance or both. The tracking algorithm can locate a desired reactance minimum within a frequency range window, which window can be adjusted as the reactance minimum shifts, so that the frequency of the reactance minimum is located within the window for rapid detection. Upon rapid load changes, if the frequency of the reactance minimum changes rapidly and falls outside of the frequency range window between tracking scans, a reset process can be executed to again locate the reactance minimum and obtain a narrow frequency range in which the reactance minimum resides for rapid determination of the frequency of the reactance minimum.

The acoustophoretic devices, including that illustrated in FIG. 1 of the present disclosure, can be used in a filter “train,” in which multiple different filtration steps are used to clarify or purify an initial fluid/particle mixture to obtain the desired product and manage different materials from each filtration step. Each filtration step can be optimized to remove a particular material, improving the overall efficiency of the clarification process. An individual acoustophoretic device can operate as one or multiple filtration steps. For example, each individual ultrasonic transducer within a particular acoustophoretic device can be operated to trap materials within a given particle range. In particular, the acoustophoretic device can be used to remove large quantities of material, reducing the burden on subsequent downstream filtration steps/stages. Additional filtration steps/stages can be placed upstream or downstream of the acoustophoretic device. Multiple acoustophoretic devices can be used as well. Desirable biomolecules or cells can be recovered/separated after such filtration/purification.

The outlets of the acoustophoretic devices of the present disclosure (e.g. clarified fluid and concentrated cells), including that illustrated in FIG. 1, can be fluidly connected to any other filtration step or filtration stage. Such filtration steps can include various methods such as depth filtration, sterile filtration, size exclusion filtration, or tangential filtration. Depth filtration uses physical porous filtration mediums that can retain material through the entire depth of the filter. In sterile filtration, membrane filters with extremely small pore sizes are used to remove microorganisms and viruses, generally without heat or (c) irradiation or exposure to chemicals. Size exclusion filtration separates materials by size and/or molecular weight using physical filters with pores of given size. In tangential filtration, the majority of fluid flow is across the surface of the filter, rather than into the filter.

Chromatography can also be used, including cationic chromatography columns, anionic chromatography columns, affinity chromatography columns, mixed bed chromatography columns. Other hydrophilic/hydrophobic processes can also be used for filtration purposes.

Desirably, flow rates through the devices of the present disclosure can be a minimum of 4.65 mL/min per cm² of cross-sectional area of the acoustic chamber. Even more desirably, the flow rate can be as high as 25 mL/min/cm², and can range as high as 40 mL/min/cm² to 270 mL/min/cm², or even higher. This is true for batch reactors, fed-batch bioreactors and perfusion bioreactors, with which the acoustophoretic devices and transducers discuss herein may be used. For example, the acoustophoretic devices may be interposed between a bioreactor and a downstream filtration device, such as those discussed above. The acoustophoretic devices may be configured to be downstream of a filtration device coupled to a bioreactor, and may be upstream of other filtration devices. In addition, the acoustophoretic devices and/or other filtration devices can be configured to have a feedback to the bioreactor.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

1. A method for controlling an acoustic system, comprising:
applying a drive signal to an acoustic transducer in the acoustic system at a series of frequencies;
receiving feedback signals from the acoustic transducer at respective frequencies;
identifying, from the feedback signals, reactance minima;
selecting a reactance minimum for continuous operation; and
applying the drive signal to the acoustic transducer at a frequency that tracks with the reactance minimum.

2. The method of claim 1, further comprising:
filling an acoustic chamber of the acoustic system with a fluid mixture that includes a material; and
conducting a global frequency scan while the fluid mixture is non-flowing.

3. The method of claim 1, further comprising cycling power while applying the drive signal to the acoustic transducer at the frequency that tracks with the reactance minimum.

4. The method of claim 3, wherein each power cycle includes a power ramp up interval, a dwell interval and a power ramp down interval.

5. The method of claim 3, further comprising flowing the fluid mixture through the acoustic chamber after cycling power.

6. The method of claim 1, further comprising selecting a tracking profile for implementing parameters used in determining the reactance minimum.

7. A controller for an acoustic transducer, comprising:
a frequency scanner configured to apply a drive signal to the acoustic transducer at a series of frequencies to generate feedback signals from the acoustic transducer;
a feedback signal sensor coupled to the transducer configured to generate a feedback signal related to operation of the acoustic transducer;
a tracking engine for determining an operating point for operating the acoustic transducer in relation to the feedback signals.

8. The controller of claim 7, further comprising a compensation model configured to provide compensation values associated with respective frequencies, the compensation values being used to compensate the feedback signal.

9. The controller of claim 8, wherein the frequency scanner is configured to obtain the compensation values prior to continuous operation of the acoustic transducer.

10. A method of determining compensation for an acoustic system, comprising:
identifying a frequency range for determining a frequency response for the acoustic system;
determining root mean square (RMS) error based on the frequency response and a frequency response model over at least a portion of the frequency range; and
identifying a value for a parameter in the frequency response model based on a reduced RMS error.

11. The method of claim 10, further comprising iterating over at least the portion of the frequency range to identify the value for the parameter in the frequency response model.

12. The method of claim 10, further comprising identifying the frequency range using a resonance frequency and an anti-resonance frequency.

13. The method of claim 10, further comprising compensating the acoustic system by subtracting the frequency response model from the frequency response.

14. A method for operating an acoustic transducer, comprising:
driving the acoustic transducer over a wide range of frequencies to identify minima and maxima impedance values for frequencies in the wide range;
driving the acoustic system at a frequency associated with an initial impedance minimum; and
tracking a modified frequency of the impedance minimum while modulating power applied to the acoustic transducer.

15. The method of claim 14, further comprising using a parameter profile for tracking the modified frequency and modulating power applied to the acoustic transducer.

16. The method of claim 15, wherein the parameter profile includes one or more of frequency step size, number of steps, frequency radius, time interval between steps, modulation of frequency range, power ramping rates, power levels, dwell times or cycle repetitions.

17. The method of claim 14, further comprising coupling the acoustic transducer to a fluid entrained with material, such that modulating power causes at least some of the material to leave an acoustic wave that is generated in the fluid by the acoustic transducer.

18. A controller for operating an acoustic transducer, comprising:
a drive controller for controlling a drive for the acoustic transducer, the drive controller being configured to provide power and frequency control signals to the drive;
a feedback processing section for processing voltage and current values derived from the acoustic transducer; and
a startup controller for determining power and frequency control signals for the drive controller in accordance with a predefined startup sequence and feedback signals generated by the feedback processing section.

19. The controller according to claim 18, wherein the predefined startup sequence includes portions for tracking minimum impedance frequency while modulating power applied to the acoustic transducer.

20. The controller according to claim 18, further comprising a parameter profile accessible to the startup controller to provide parameters for the predefined startup sequence.
